(19) United States
(12) Plant Patent Application Publication
(10) Pub. No.: US 2005/0204442 P1
(43) Pub. Date: Sep. 15, 2005

(54) Chrysanthemum plant named 'Cetwotone Pink'

(76) Inventor: Mark Roland Boeder, The Hague (NL)

Correspondence Address: PARKHURST & WENDEL, SUITE 210, 1421 PRINCE STREET, ALEXANDRIA, VA, 22314, US

(21) Appl. No.: 10/782,794

(22) Filed: Feb. 23, 2004

(51) Int. Cl. A01H 5/00

(52) U.S. Cl. PLT/297

(57) ABSTRACT

A Chrysanthemum plant named ‘Cetwotone pink’ characterized by its bicolored medium sized blooms with purple-white ray-florets and green disc florets, with a response time of 49 days.

### BACKGROUND OF THE INVENTION

‘Cetwotone Pink’ is a product of a breeding-program which had the objective of creating new chrysanthemum cultivars with a daisy type flower, a 7 week response and a medium plant height (1 m). The new plant of the present invention comprises a new and distinct cultivar of Chrysanthemum plant. ‘Cetwotone Pink’ is a seedling from a cross in a breeding program maintained under the control of inventor. The female parent is #97.0243-unpatented-, an unnamed seedling not available to inventor for description. The male parent is unknown, being a mixed population of a group of male parents. The new and distinct cultivar was discovered and selected as a flowering plant within the progeny of the stated cross by Mark Roland Boeder in a controlled environment (greenhouse) in Rijsenhout Holland in 2001. The first act of asexual reproduction of ‘Cetwotone Pink’ was accomplished when vegetative cuttings were taken from the initial selection in 2001 in a controlled environment in Rijsenhout Holland.

### SUMMARY OF THE INVENTION

The present invention is a new and distinct variety of chrysanthemum bearing medium sized blooms with bicolored purple-white ray-florets and green disc florets.

### BRIEF DESCRIPTION OF THE DRAWINGS

The present invention of a new and distinct variety of chrysanthemum is shown in the accompanying drawings, the color being as nearly true as possible with color photographs of this type.

FIG. 1 shows a plant of the cultivar in full bloom.

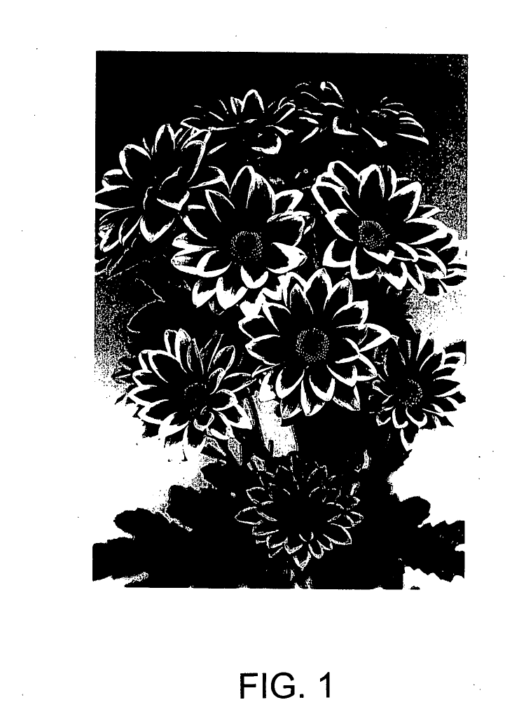

FIG. 2 shows the various stages of bloom of the new cultivar.

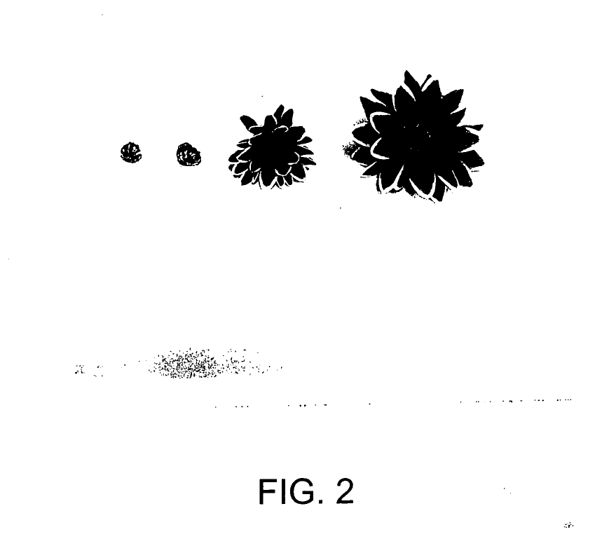

FIG. 3 shows the foliage of the new cultivar.

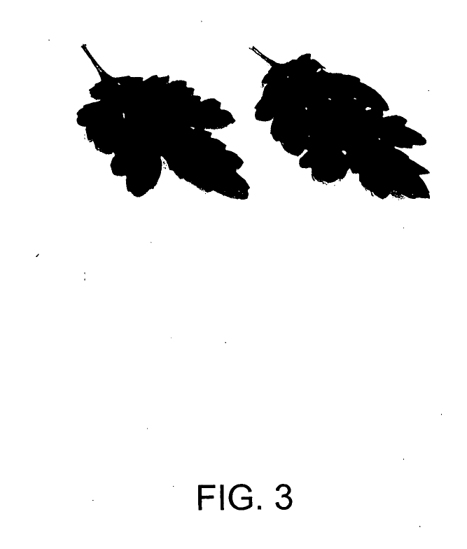

### DESCRIPTION OF THE INVENTION

This new variety of chrysanthemum is of the botanical classification Chrysanthemum morifolium. The observations and measurements were gathered from plants grown in a greenhouse in Rijsenhout Holland in a photo-periodic controlled crop under conditions generally used in commercial practice. The greenhouse temperatures during this crop were at day-time between 18° C. and 25° C. and at night 20° C. After a long day period of 14 days the photo-periodic response time in this crop was 49 days. After the long day period to flowering growth retardants were applied 2 to 3 times in an average dose of 2.5 gram/liter water. The plants were observed (directly) during the flowering of this crop. No tests were done on disease or insect resistance or susceptibility. No tests were done on cold or drought tolerance. This new variety produces medium sized blooms with purple-white ray-florets and green disc-florets blooming on the plant for 5 weeks. This new variety of chrysanthemum has been found to retain its distinctive characteristics throughout successive propagations however the phenotype may vary significantly with variations in environment such as light intensity and temperature. To show the phenotype as described ‘Cetwotone pink’ can be planted without assimilation lightning (high pressure sodium lamps) between week 4 and week 32 under greenhouse conditions in Holland. With assimilation light (minimum level 2500 lux) it can be planted year round under greenhouse conditions in Holland.

From the cultivars known to inventor the most similar existing cultivar in comparison to ‘Cetwotone Pink’ is ‘Orinoco’ (U.S. Plant Pat. No. 14,338). When ‘Orinoco’ and ‘Cetwotone pink’ are being compared the following differences and similarities are noticed: The differences of ‘Orinoco’ and ‘Cetwotone Pink’ are (1) Flower size. The flowers of ‘Orinoco’ are larger in size than those of ‘Cetwotone Pink’ (2) Number of inflorescences. ‘Orinoco’ has a smaller number of inflorescences per stem than ‘Cetwotone Pink’. (3) Number of ray florets per inflorescences. The inflorescences of ‘Orinoco’ have a larger number of ray florets than ‘Cetwotone Pink’.

The following is a description of the plant and characteristics that distinguish ‘Cetwotone Pink’ as a new and distinct variety. The color designations are taken from the plant itself. Accordingly, any discrepancies between the color designations and the colors depicted in the photographs are due to photographic tolerances. The color chart used in this description is: The Royal Horticultural Society Colour Chart, edition 1995.

TABLE 1
Botanical Description of culitvar ‘Cetwotone Pink’

| | |
|---|---|
| Bud | |
| Size | Medium, cross-section 0.8 cm |
| | height 0.8 cm |
| Outside color | Green 143C |
| Involucral bracts | 2 rows, length 7 mm, width 3 mm |
| Involucral bracts among disc-florets | Not present |
| Involucral bracts color | Green 139B |
| Inflorescence | |
| Type | Single |
| Height | 1.5 cm |
| Size | medium |
| Fully expanded | 4.5 cm |
| Number of blooms per stem | Average of 15 |
| Performance on the plant | 5 weeks |
| Seeds (if crossed) | Produced in large quantities, |
| | oval shaped, grey-brown 199A, |
| | 2 mm in length. |
| Fragrance | Typical chrysanthemum, slight |
| Peduncle length | Near the top 10 cm, near the |
| | middle 11 cm, near the |
| | bottom 12 cm |
| Peduncle color | Green 138B |
| Color | |
| Center of the flower (disc-florets) | Immature Green 143C |
| | Mature Yellow 7A |
| Color of the ray-florets | Upper surface Red-purple 71A and |
| | White 155D at edges |
| | Lower surface Greyed-purple 186A |
| | and White 155D |
| Tonality from Distance | A spray mum with bicolored |
| | purple-white flowers and a |
| | green disc |
| Color of the upper surface of the | Red-purple 71A and White 155D |
| ray-florets after aging of the plant | |
| Ray florets | |
| Texture | Upper and under side smooth |
| Number | 20-25 |
| Cross-section | Flat |
| Longitudinal axis of majority | Straight |
| Length of corolla tube | Short; 0.2 cm |
| Ray-floret margin | Entire |
| Ray-floret length | 2 cn |
| Ray-floret width | 0.7 cm |
| Ratio length / width | Medium |
| Shape of tip | Rounded |
| Disc florets | |
| Disc diameter | 1 cm |
| Distribution of disc florets | Numerous, clearly visible at all |
| | stages of flowering. |
| Shape | Tubular |
| Color | Yellow-green 154D |
| Receptacle shape | Domed raised |
| Reproductive Organs | |
| Stamen | Present in disc florets only |
| Stamen color | Yellow-green 144 B |
| Pollen | Produced in large quantities |
| Pollen color | Yellow 7A |
| Styles | Present in both ray and disc florets |
| Style color | Yellow 13A |
| Style Length | 4 mm |
| Stigma color | Yellow-green 145D |
| Stigma Width | 1 mm |
| Ovaries | Enclosed in perianth |
| Plant | |
| Form | A spray mum meant for erect |
| | culture |
| Growth habit | Upright |
| Growth rate | Medium vigour |
| Height | 90 cm |
| Width | 20 cm |
| Internode lenght | 3 cm |
| Spray formation | Corymbiform |
| Stem Color | Green 138B to 143A with streaks |
| | of Greyed-purple 185D |
| Stem Strength | Medium |
| Stem Brittleness | Not brittle |
| Stem Anthocyanin coloration | Present |
| Flowering Response (photo- | 49 days |
| periodic controlled crop, not natural | |
| season) | |
| Foliage | |
| Color immature stage | Upper side Green 137C |
| | Under side Green 138B |
| Color mature stage | Upper side Green 137A |
| | Under side Green 138A |
| Color midvein mature leaf | Upper side Green 138C |
| | Under side Yellow-green 147G |
| Size | Large; length 14 cm, width 7 cm |
| Quantity (number per single stem) | 25 |
| Shape | Elliptic |
| Texture upper side | Fleshy and glabrous |
| Texture under side | Pubescent |
| Venation arrangement | Palmate |
| Shape of the margin | Serrate |
| Shape of Base of Sinus Between | Rounded |
| Lateral Lobes | |
| Margin of Sinus Between Lateral | Diverging |
| Lobes | |
| Shape of Base | Obtuse-rounded |
| Apex | Micronulate |
| Petiole Lenght | 2 cm |
| Petiole Color | Green 138C |

TABLE 2
Differences with the comparison varieties

| | ‘Cetwotone Pink’ | ‘Orinoco’ |
|---|---|---|
| Diameter infloresences | 4.5 cm | 7.5 cm |
| Number inflorescences per stem | 25-30 | 17 |
| Number ray florets per stem | 20-25 | 27-40 |

1. A new and distinct variety of chrysanthemum plant as described and illustrated.
